(19) United States
(12) Patent Application Publication
(10) Pub. No.: US 2010/0074914 A1
(43) Pub. Date: Mar. 25, 2010

(54) DNA vaccine comprising CTGF-encoding DNA construct and applications thereof

(75) Inventors: Wen-Fang Cheng, Taipei City (TW); Chi-An Chen, Taipei City (TW)

Correspondence Address: BACON & THOMAS, PLLC, 625 SLATERS LANE, FOURTH FLOOR, ALEXANDRIA, VA, 22314-1176, US

(73) Assignee: National Taiwan University, Taipei City (TW)

(21) Appl. No.: 12/232,631

(22) Filed: Sep. 22, 2008

(51) Int. Cl. A61K 39/12; C12Q 1/68; A61K 9/14; A61P 35/04

(52) U.S. Cl. 424/186.1; 435/320.1; 424/493

(57) ABSTRACT

The present invention provides a DNA vaccine, which comprises a DNA construct comprising an expression vector which is expressible in a eukaryotic cell, and a nucleotide fragment which comprises a CTGF-encoding sequence and a HPV sequence selected from an E6-encoding sequence, an E7-encoding sequence, or a combination thereof. In addition, the present invention also provides a pharmaceutical composition and a method of generating said DNA vaccine.

### BACKGROUND OF THE INVENTION

1. Field of the Invention

The present invention relates to a DNA vaccine comprising a DNA construct. The present invention also relates to a pharmaceutical composition and a method of generating said DNA vaccine.

2. Description of the Related Art

Cervical cancer is a main cause of death in women, and persistent infection with known high-risk Human papillomavirus (HPV) types, such as HPV subtype 16 (HPV-16) is directly linked to development and progression of cervical cancer. Although the present HPV vaccines can be used to prevent cervical cancer, it is not yet known whether they are useful for treating HPV-associated cervical cancer or ameliorating the existing HPV infection or HPV-associated lesions. Therefore, it is advantageous to develop a safe and effective therapeutic vaccine for treatment of HPV-associated cervical cancer, HPV-associated other genital cancers (e.g., vaginal, vulvar, penile) and other HPV-associated lesions of head and neck, and gastrointestinal system, as well as for the treatment of precancerous lesions of the same areas.

In recent years, DNA vaccines have been developed and evaluated in a variety of disease models as promising therapeutic agents for treating a variety of diseases including their use as immunotherapy in certain cancers. Therapeutic DNA vaccines are introduced into antigen-presenting cells (APCs) of the recipient's immune system to express a protein antigen, the protein antigen is processed and presented by the major histocompatibility antigen (MHC) class I and II molecules to induce immune responses, such as helper T-cell response, cytotoxic T-cell response and humoral (antibody) response, and a direct result is that tumor cells expressing said antigen can be eliminated by the immune system.

In comparison with “traditional” (protein antigen) vaccines, DNA vaccines have many advantages such as high specificity, safety, stability, cost-effectiveness, and the ability to induce several types of immune responses. DNA vaccines do not pose any risk of infection because only certain sequences of the pathogen's DNA is used in their manufacture, in contrast to “live” and “attenuated” vaccines. Also, no toxic adjuvants are needed for practical use of DNA vaccines. DNA vaccines are also easier to prepare than “subunit” vaccines because no protein antigens need to be expressed and purified prior to injection into the patient.

However, because the lifespan of APCs is limited, the efficacy of DNA vaccines is limited because APCs are not able to process and present the antigen indefinitely. Therefore, several strategies have been applied to increase the potency of DNA vaccines, such as targeting an antigen by fusing molecules to enhance antigen processing (Cheng et al., 2001; Chen et al., 2000), targeting antigens for rapid intracellular degradation (Rodriguez et al., 1997), directing antigens to APCs by fusion to ligands for APC receptors (Boyle et al., 1998) or to a pathogen sequence such as fragment C of tetanus toxin (King et al., 1998), co-injecting cytokines (Weiss et al., 1998), and administering with CpG oligonucleotides (Klinman et al., 1997).

Combined strategies for enhancing the effects of DNA vaccines have been introduced in the development of cancer vaccines and immunotherapy, and overexpression of an anti-apoptotic molecule is a potential strategy for overcoming the short lifespan of APCs. For example, the administration of a DNA vaccine which comprises fragments encoding an antigen and an inhibitor of apoptosis of dendritic cells (DCs) can prolong the survival of DCs, thereby enhancing the potency of the DNA vaccine (Kim et al., 2003). Other research has demonstrated that combining a tumor antigen and an apoptosis inhibitor, such as Bcl-X1, Bcl-2, XIAP, dominant negative caspase-9, or dominant negative caspase-8, can enhance the antigen-specific immunity and the anti-tumor effects (Kim et al., 2003; Kim et al., 2004; Kim et al., 2005). Thus, DC-based vaccination could be enhanced by an approach that inhibits apoptosis and prolongs the survival of antigen-expressing DCs in vivo. However, some apoptosis inhibitors, such as proteins of Bcl-2 family, are known to be over-expressed in some cancers and therefore they are implicated in contributing to cellular immortalization. This safety issue cannot be ignored.

### SUMMARY OF THE INVENTION

In view of the disadvantages of conventional DNA vaccines, the objective of the present invention is to develop a DNA vaccine having an improved potency, which comprises a sequence obtained from the connective tissue growth factor (CTGF) gene and a sequence obtained from E6 and/or E7 gene of Human Papillomaviruses. The combination of CTGF and a HPV sequence selected from E6 and/or E7 enhances the recipient's immune responses and create a more potent anti-tumor effect.

Another objective of the present invention is to provide a pharmaceutical composition comprising said DNA vaccine and an optional pharmaceutically acceptable carrier. Yet another objective of the present invention is to provide a method of generating said DNA vaccine.

To achieve the above-mentioned objectives, the present invention provides a DNA construct, comprising:
an expression vector, which is expressible in a eukaryotic cell; and
a nucleotide fragment, which comprises a CTGF-encoding sequence, and a HPV sequence selected from an E6-encoding sequence, an E7-encoding sequence, or a combination thereof.

Said E6-encoding sequence and said E7-encoding sequence can be derived from all HPV subtypes, particularly, from HPV-16.

In preferred embodiments, said expression vector is expressible in human cells; more preferably, said expression vector is selected from pcDNA3, pSG5, or pCMV; and most preferably, said expression vector is pcDNA3.

In preferred embodiments, said CTGF-encoding sequence is SEQ ID NO: 3, said E6-encoding sequence is SEQ ID NO: 6, and said E7-encoding sequence is SEQ ID NO: 9.

The present invention also provides a DNA vaccine, comprising:
the above-mentioned DNA construct; and
a particle, which is coated with said DNA construct.

In preferred embodiments, said particle is a gold particle; more preferably, said gold particle has a diameter of 1.6 μm.

In preferred embodiments, wherein said expression vector is expressible in human cells; more preferably, said expression vector is selected from pcDNA3, pSG5, or pCMV; and most preferably, said expression vector is pcDNA3.

In preferred embodiments, said CTGF-encoding sequence is SEQ ID NO: 3, said E6-encoding sequence is SEQ ID NO: 6, and said E7-encoding sequence is SEQ ID NO: 9.

The present invention even provides a pharmaceutical composition, comprising the above-mentioned DNA vaccine.

In preferred embodiments, said pharmaceutical composition further comprises a pharmaceutically acceptable carrier; more preferably, said pharmaceutically acceptable carrier is ddH₂O or PBS (phosphate buffered saline).

In preferred embodiments, said pharmaceutical composition is used to treat HPV-induced diseases; more preferably, used to treat a genital cancer (e.g., cervical cancer, vaginal cancer, vulvar cancer, penile cancer) or a precancerous lesion (e.g., cervical, vulvar, or vaginal precancerous lesion), a head and neck cancer (e.g., oropharyngeal squamous cell carcinoma), or a gastrointestinal cancer (e.g., esophageal cancer, or colorectal cancer); most preferably, used to treat cervical cancer.

The present invention yet provides a method of generating the above-mentioned DNA vaccine, comprising:

(1) providing a DNA construct comprising:
an expression vector, which is expressible in a eukaryotic cell; and
a nucleotide fragment, which comprises a CTGF-encoding sequence, and a HPV sequence selected from an E6-encoding sequence, an E7-encoding sequence, or a combination thereof; and

(2) coating said DNA construct on the surface of a particle.

In preferred embodiments, said expression vector is pcDNA3; said CTGF-encoding sequence is SEQ ID NO: 3, said E6-encoding sequence is SEQ ID NO: 6, and said E7-encoding sequence is SEQ ID NO: 9; more preferably, said particle is a gold particle; and most preferably, said gold particle has a diameter of 1.6 μm.

The present invention further provides a method for preventing or treating a HPV-induced disease, comprising:

administering an effective amount of the above-mentioned DNA vaccine or an effective amount of the above-mentioned pharmaceutical composition to a subject having or at risk for developing said HPV-induced disease.

In preferred embodiments, said HPV-induced disease is a genital cancer or precancerous lesion, a head and neck cancer, or a gastrointestinal cancer; more preferably, said genital cancer comprises cervical cancer, vaginal cancer, vulvar cancer, and penile cancer; said genital precancerous lesion comprises cervical, vulvar, and vaginal precancerous lesions; said head and neck cancer comprises oropharyngeal squamous cell carcinoma, and said gastrointestinal cancer comprises esophageal cancer, colorectal and anal cancer and precancerous lesions of the same; most preferably, said HPV-induced disease is cervical cancer.

In summary, the present invention provides a DNA vaccine, which comprises a DNA construct comprising an expression vector which is expressible in a eukaryotic cell, and a nucleotide fragment which comprises a CTGF-encoding sequence and a HPV sequence selected from an E6-encoding sequence and/or an E7-encoding sequence; which has an excellent potency and can be used for the treatment of HPV-induced diseases. Also, the present invention provides a pharmaceutical composition and a method of generating said DNA vaccine.

### BRIEF DESCRIPTION OF THE DRAWINGS

FIG. 1 is the schematic diagram of the DNA constructs.

FIG. 2 shows the results of the E7-specific immunological profiles of vaccinated mice. (a) Representative figures from the flow cytometric analysis of E7-specific IFN-γ-secreting CD4⁺ T lymphocytes in each group. (b) Bar graph depicting the number of E7-specific IFN-γ-secreting CD8⁺ T lymphocytes per 3.5×10⁵ splenocytes (mean±SEM, P<0.01, one-way ANOVA). (c) Bar graph depicting the number of E7-specific IFN-γ-secreting CD4⁺ T lymphocytes per 3.5×10⁵ splenocytes (mean±SEM, P>0.05, one-way ANOVA). (d) Bar graph depicting the number of E7-specific IL-4-secreting CD4⁺ T lymphocytes per 3.5×10⁵ splenocytes (mean±SEM, P<0.01, one-way ANOVA). (e) Bar graph showing the titers of anti-E7 antibodies in mice vaccinated with various DNA vaccines (mean±SEM, P<0.01, one-way ANOVA).

FIG. 3 shows the results of the E7-specific immunological profiles of mice vaccinated with various DNA vaccines: CTGF/E7, CTGF/E6, CTGF/E6/E7, CTGF/E6+CTGF/E7. (a) Representative figures from the flow cytometric analysis of E6-specific IFN-γ-secreting CD8⁺ T lymphocytes in each group. (b) Representative figures from the flow cytometric analysis of E7-specific IFN-γ-secreting CD8⁺ T lymphocytes in each group. (c) Bar graph depicting the number of E6-specific and/or E7-specific IFN-γ-secreting CD8⁺ T lymphocytes per 3.5×10⁵ splenocytes (mean±SEM, P<0.01, one-way ANOVA).

FIG. 4 shows the results of in vivo tumor protection experiments in mice. (a) In vivo tumor protection experiments in mice vaccinated with various DNA vaccines: naïve, no insert, E7, CTGF, E7+CTGF, CTGF/E7. (b) In vivo tumor protection experiments in mice vaccinated with various DNA vaccines: naïve, E7, CTGF/E6, CTGF/E7, CTGF/E6/E7, CTGF/E6+CTGF/E7.

FIG. 5 shows the results of in vivo antibody depletion experiments in mice.

FIG. 6 shows in vivo tumor treatment in mice using a high therapeutic dose. (a) Representative pulmonary tumor nodules of each vaccinated group. 1: naïve, 2: no insert, 3, E7, 4: CTGF, 5, CTGF/E7. (b) The number of pulmonary tumor nodules of each vaccinated group (mean±SEM, P<0.001, one-way ANOVA).

FIG. 7 shows flow cytometric analysis of DNA-transfected dendritic cells in the inguinal lymph nodes of vaccinated mice and activation of E7-specific CD8⁺ T cells isolated from the inguinal lymph nodes of vaccinated mice. (a) Bar graph depicting the percentage of CD11c⁺ GFP⁺ monocytes among the gated monocytes (mean±SEM, P<0.001, one-way ANOVA). (c) Bar graph depicting the percentage of apoptotic cells in CD11c⁺ GFP⁺ cells (mean±SEM, P<0.001, one-way ANOVA). (d) Bar graph depicting the percentage of E7-specific IFN-γ-secreting CD8⁺ T cell per 1×10⁵ cells (mean±SEM, P<0.001, one-way ANOVA).

### DETAILED DESCRIPTION OF THE PREFERRED EMBODIMENTS

HPV early genes are potentially useful as target antigens for therapeutic HPV vaccines because these early genes are expressed throughout the life cycle of HPV. The HPV early genes E6 and E7 (also known as oncogenes because of their ability to transform the host cells) are especially desirable for use in DNA vaccines to treat HPV-induced diseases, including genital, head and neck, and gastrointestinal cancers and related precancerous lesions. However, many DNA vaccines lack sufficient immunogenicity to render them useful because the DNA comprising the vaccines cannot amplify or spread in vivo, and antigen processing and presentation plays an important role in the production of useful therapeutic DNA vaccines.

Connective tissue growth factor (CTGF) is a cysteine-rich protein originally identified in a conditioned medium of human umbilical vein endothelial cells. Recently, CTGF has been shown to inhibit apoptosis of chicken embryo fibroblasts and human rhabdomyosarcoma cells. It has also been demonstrated to promote endothelial cell survival. CTGF can also promote the acquisition of the apoptosis-resistant phenotype that may be similar to those of IGF-II in skeletal myoblasts.

The following examples are used to further illustrate the importance of the present invention, and are not intended to limit the claims of the invention. Of particular importance is that “E6” and “E7” as stated in this invention refer to the E6 and E7 gene of ANY subtype of Human Pappilomavirus.

### EXAMPLES

### DNA Constructs and Preparation

CTGF was amplified by polymerase chain reaction (PCR) using human placenta complementary DNA as the template and a set of primers, namely, 5′-CCGGTCTAGACAACCATGACCGCCGCCAGT-3′ (SEQ ID NO: 1) and 5′-CCGGAATTCGTTCAAGTTCCAGTCTAATG-3′ (SEQ ID NO: 2). The amplified CTGF nucleotide sequence (SEQ ID NO: 3) was then cloned into the XbaI/EcoRI sites of pcDNA3 vector (Invitrogen, Carlsbad, Calif., USA) to generate pcDNA3-CTGF.

E6 was amplified by PCR using the DNA of the CaSki cell line (a cell line containing an integrated HPV 16 genome, obtained from ATCC) as the template and with a set of primers, 5′-GGGGAATTCATGCACCAAAAGAGAACTGCAATGT-3′ (SEQ ID NO: 4) and 5′-CCCAAGCTTTTACAGCTGGGTTTCTCTACGTGTTC T-3′ (SEQ ID NO: 5). The amplified E6 nucleotide sequence (SEQ ID NO: 6) was then cloned into the EcoR1/HindIII sites of pcDNA3 and pcDNA3-CTGF to generate pcDNA3-E6 and pcDNA3-CTGF/E6, respectively. In addition, E7 was amplified by PCR using the DNA of the CaSki cell line as the template and with a set of primers, 5′-CCGGAAGCTTATGCATGGAGATACACCTAC-3′ (SEQ ID NO: 7) and 5′-CCCAAGCTTTTGAGAACAGATGG-3′ (SEQ ID NO: 8). The amplified E7 nucleotide sequence (SEQ ID NO: 9) was then cloned into the HindIII sites of pcDNA3 and pcDNA3-CTGF to generate pcDNA3-E7 and pcDNA3-CTGF/E7, respectively.

To generate pcDNA3-CTGF/E6/E7, the amplified E7 nucleotide sequence (SEQ ID NO: 9) was cloned into the HindIII sites of pcDNA3-CTGF/E6.

Said pcDNA3-CTGF/E6 contains the CTGF and E6 nucleotide sequences joined in a continuous open reading frame such that a fusion protein comprised of CTGF and E6 is expressed by the constructs. Similarly, pcDNA3-CTGF/E7 can produce a fusion protein comprised of CTGF and E7, and pcDNA3-CTGF/E6/E7 can produce a fusion protein comprised of CTGF, E6 and E7.

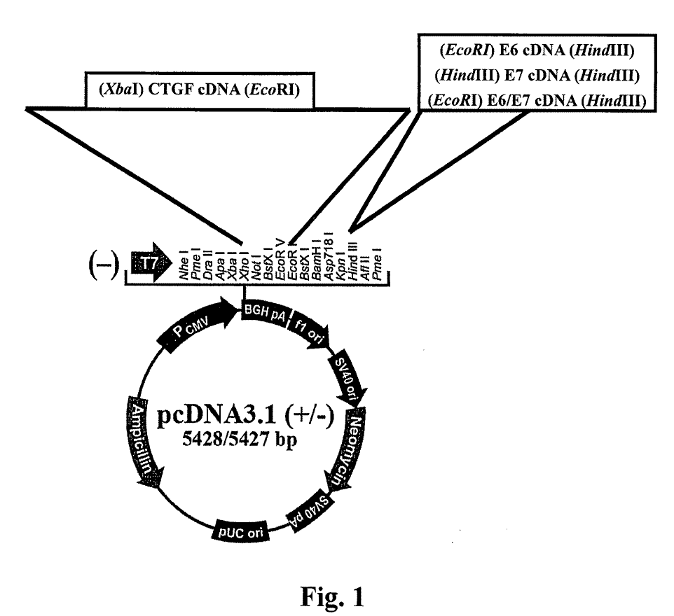

Next, pcDNA3-CTGF/E7 was digested by HindIII, and then the GFP fragment obtained from the plasmid pcDNA3-E7/GFP (a gift from Dr. T C Wu of Johns Hopkins Medical Institutes) by EcoRI/NotI digestion filled and ligated to generate pcDNA3-CTGF/E7/GFP. All DNA constructs were confirmed by DNA sequencing, and the schematic diagram of the DNA constructs is shown in FIG. 1.

### Mice

Six- to eight-week-old female C57BL/6J mice were used in groups of five in all of the subsequently described studies. All animal procedures were performed according to approved protocols and in accordance with recommendations for the proper use and care of laboratory animals at the animal care facility of the College of Medicine, National Taiwan University.

### DNA Vaccination

Preparation of DNA-coated gold particles and particle-mediated DNA vaccination using a helium-driven gene gun were performed by a low pressure-accelerated gene gun (BioWare Technologies Co. Ltd., Taipei, Taiwan). The gold particles (Bio-Rad 1652263) were weighted and suspended in 70% ethanol. This suspension was vortexed vigorously and then centrifuged to collect the particles. After washing with distilled water three times, 0.025 μg of the collected gold particles were put in an Eppendorf tube and mixed with 100 μL of 0.05 M spermidine by vortexing, and the mixture was sonicated for 10 to 20 seconds. Subsequently, 25 μg of DNA dissolved in 25 μL of ddH₂O was added and the mixture was vortexed. 100 μL of 1 M CaCl₂ was added, and the final mixture was vortexed and incubated on ice for 10 minutes to coat DNA on the gold particles via spermidine. Finally, the coated particles were washed with 100% ethanol three times and resuspended in 200 to 250 μL of 100% ethanol. The DNA-coated gold particle suspension was used as a bullet for the gene gun. Naked DNA dissolved in ddH₂O or PBS also can be used as a bullet. DNA-coated gold particles were delivered to the shaved abdominal region of mice using the low pressure-accelerated gene gun with a 50 psi discharge pressure of helium.

### Intracellular Cytokine Staining and Flow Cytometric Analysis

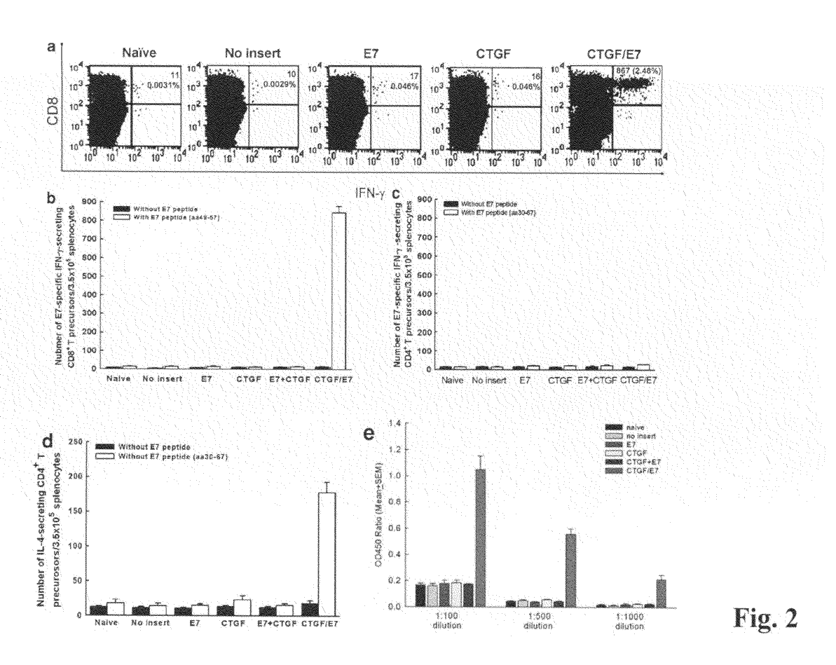

First of all, each group of mice was immunized with one of the following DNA vaccines: pcDNA3 (no insert), pcDNA3-E7, pcDNA3-CTGF, pcDNA3-E7+pcDNA3-CTGF and pcDNA3-CTGF/E7 (2 μg DNA construct/2 μg gold particle/mouse); all mice received a booster immunization one week later. A DNA-naïve group, in which the mice were not vaccinated, was used as a negative control. One week after boosting, the mice were sacrificed and their splenocytes were harvested and incubated with either 1 μg/ml of short E7 peptide RAHYNIVTF (aa 49-57, SEQ ID NO: 10) or 10 μg/ml of long E7 peptide DSSEEEDEIDGPAGQAEPDRA HYNIVTFCCKCDSTLRL (aa 30-67, SEQ ID NO: 11). Generally speaking, the short E7 peptide could be presented directly; however, the long E7 peptide required up-take by the APCs first for subsequent processing and presentation. The cells were mixed with the short or long E7 peptide for 16 to 20 hours. Next, 1 μL/mL Golgistop (PharMigen, San Diego, Calif.) was added to prevent the secretion of cytokines, such as IFN-γ or IL-4. Six hours later, cells were harvested, transferred into a tube, and then centrifuged at 1,200-1,600 rpm for 5 min at 4° C. Next, the cells were washed with 500 μL FACScan buffer (0.5% BSA in PBS), and re-centrifuged for 5 min at 4° C. The cells were re-suspended in 1 μL (0.5 ug) PE-conjugated anti-CD4 or anti-CD8 Ab (PharMingen) diluted in 50 μL FACScan buffer, and the cells, were incubated for 30 minutes in the dark. The cells were then washed twice with FACScan buffer and centrifuged. These cells were re-suspended in 500 μL fixation buffer on ice for 20 minutes in the dark; subsequently, the cells were again centrifuged and washed with 500 μL Perm Wash buffer (BioLegend Biotech). 1 μL (0.5 ug) FITC-conjugated anti-IFN-γ Ab (PharMingen) or anti-IL-4 Ab (Biolegend, San Diego, Calif.) was diluted in 50 μL Perm Wash buffer, added to the cells, and incubated on ice for 30 minutes in the dark. The cells were centrifuged and washed twice with 500 μL Perm Wash buffer. Then the cells were resuspended in 300 to 500 μL FACScan buffer, and analyzed by flow cytometry. All double-stained cells are analyzed by FACScan or FACSCalibur with CELLQuest software (Becton Dickinson Immunocytometry System, Mountain View, Calif., USA) using a standard procedure. The results are shown in FIG. 2.

FIG. 2a shows the flow cytometric analysis of E7-specific IFN-γ-secreting CD8⁺ T lymphocytes in various vaccinated groups. FIGS. 2b and 2c are the bar graphs depicting the number of E7-specific IFN-γ-secreting CD8⁺ T lymphocytes and E7-specific IFN-γ-secreting CD4⁺ T lymphocytes per 3.5×10⁵ splenocytes, respectively. In addition, FIG. 2d shows the bar graph depicting the number of E7-specific IL-4-secreting CD4⁺ T lymphocytes per 3.5×10⁵ splenocytes. These data demonstrate that the group of mice vaccinated with CTGF/E7 generated more E7-specific IFN-γ-secreting CD8⁺ T lymphocytes and E7-specific IL-4-secreting CD4⁺ T lymphocytes than other groups. In other words, CTGF/E7 could activate cytotoxic T lymphocytes (FIG. 2b) and Th2 pathway (FIG. 2d), but not Th1 pathway (FIG. 2c).

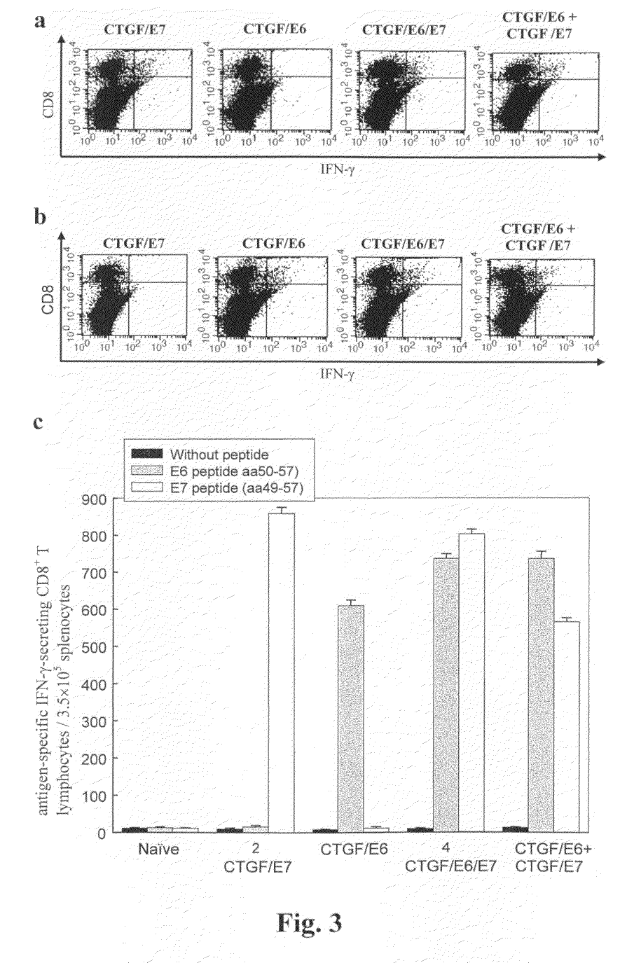

Similarly, other five groups of mice was immunized and sacrificed as above except using the following DNA vaccines: pcDNA3-CTGF/E7, pcDNA3-CTGF/E6, pcDNA3-CTGF/E6/E7, and pcDNA3-CTGF/E6+pcDNA3-CTGF/E7. Then the splenocytes of these mice were harvested and incubated with either 1 μg/ml of short E7 peptide (i.e. SEQ ID NO: 10), or 1 μg/ml of short E6 peptide LCIVYRDG (aa 50-57, SEQ ID NO: 12) for 16 to 20 hours, and treated as above to identify the number of E6 and/or E7 antigen-specific IFN-γ-secreting CD8⁺ T lymphocytes per 3.5×10⁵ splenocytes. FIGS. 3a and 3b are the flow cytometric analysis of E7-specific IFN-γ-secreting CD8⁺ T lymphocytes and of E6-specific IFN-γ-secreting CD8⁺ T lymphocytes in the above-mentioned vaccinated groups, respectively. In FIG. 3c, E7-specific IFN-γ-secreting CD8⁺ T lymphocytes increased in pcDNA3-CTGF/E7, pcDNA3-CTGF/E6/E7, and pcDNA3-CTGF/E6+pcDNA3-CTGF/E7 groups; and E6-specific IFN-γ-secreting CD8⁺ T lymphocytes increased in pcDNA3-CTGF/E6, pcDNA3-CTGF/E6/E7, and pcDNA3-CTGF/E6+pcDNA3-CTGF/E7 groups. This data shows that a physical linkage of CTGF to E6 or E7 was required for the enhancement of E6- or E7-specific IFN-γ-secreting CD8⁺ T lymphocytes activity. That is to say, CTGF/E6/E7 could also activate cytotoxic T lymphocytes. Similarly, it could activate Th2 pathway, but not Th1 pathway (data not shown)

### Enzyme-Linked Immunosorbent Assay (ELISA) for Anti-E7 Antibodies

Sera from all mice were extracted 14 days after the last immunization and were used to detect HPV-16 E7-specific antibodies by the direct enzyme-linked immunosorbent assay described below.

A 96-microwell plate was coated with 100 μL of bacteria-derived HPV-16 E7 protein (0.5 μg/ml) and incubated at 4° C. overnight. The wells were then blocked with PBS containing 20% fetal bovine serum at 37° C. for 2 hours. 100 μL of the sera at various dilutions of 1:100, 1:500, or 1:1,000 in PBS was then added and the plate was incubated at 37° C. for 2 hours. The wells were then washed with PBS containing 0.05% Tween 20, and then incubated with a 1:2000 dilution of peroxidase-conjugated rabbit anti-mouse IgG antibody (Zymed, San Francisco, Calif.) at room temperature for 1 hour. The plate was washed, developed with 1-Step Turbo TMB-ELISA (Pierce, Rockford, Ill.), and the reaction was stopped with 1M H₂SO₄. The ELISA plate was read with a standard ELISA reader at 450 nm.

FIG. 2e shows E7-specific antibodies in mice vaccinated with the various DNA vaccines. Mice vaccinated with the CTGF/E7 DNA vaccine had a titer higher than those in the other groups.

In vivo Tumor Protection Experiments

To determine whether the observed enhancement of E7-specific T cell immunity can be translated into a significant E7-specific protective anti-tumor effect, we performed the following in vivo tumor protection experiments. Each group of mice was immunized with pcDNA3 (no insert), pcDNA3-E7, pcDNA3-CTGF, pcDNA3-E7+pcDNA3-CTGF or pcDNA3-CTGF/E7 (2 μg/mouse), and all mice received a booster one week later. Mice were challenged with 5×10⁴ TC-1 cells/mouse by subcutaneous injection one week after the boosting. These mice were monitored until 60 days after TC-1 challenge. The naïve group, in which the mice were not vaccinated, was used as a negative control.

TC-1 cells were grown in RPMI-1640 supplemented with 10% (vol/vol) fetal bovine serum, 50 units/mL penicillin/streptomycin, 2 mM L-glutamine, 1 mM sodium pyruvate, 2 mM non-essential amino acids (Gibco company) and 0.4 mg/mL G418 at 37° C. with 5% CO₂. On the day of tumor challenge, TC-1 cells were harvested by trypsinization, washed twice with 1×PBS and finally re-suspended in 1×Hanks buffered salt solution for TC-1 challenge.

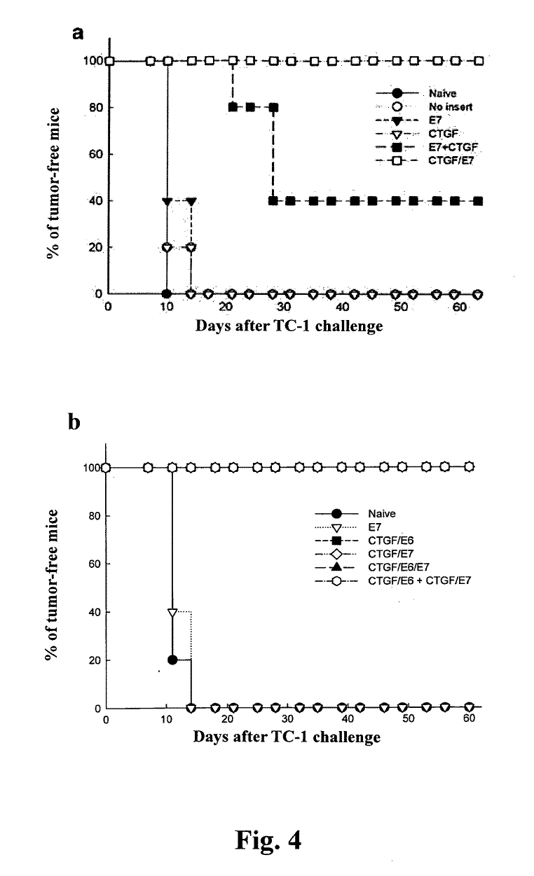

After challenging with TC-1 tumor cells, 100% of the mice that received the CTGF/E7 DNA vaccine remained tumor-free at day 60, and all mice in the other groups, including the E7 group, developed tumors within 14 days after TC-1 challenge. The results are shown in FIG. 4a.

Similarly, as shown in FIG. 4b, other five groups of mice was immunized and challenged as above except using the following DNA vaccines: pcDNA3-E7, pcDNA3-CTGF/E6, pcDNA3-CTGF/E7, pcDNA3-CTGF/E6/E7, and pcDNA3-CTGF/E6+pcDNA3-CTGF/E7. 100% of the mice that received the CTGF/E6, CTGF/E7, CTGF/E6/E7, or CTGF/E6+CTGF/E7 vaccine remained tumor-free at day 60. These results indicate that fusion of CTGF to antigen E6 and/or E7 is required for anti-tumor immunity of E6- and/or E7-expressing TC-1 tumor cells.

In vivo Antibody Depletion Experiment

To determine whether the subsets of lymphocytes (CD4⁺ T lymphocytes, CD8⁺ T lymphocytes and NK lymphocytes) are important for the anti-tumor effects, we performed an in vivo antibody depletion experiment. The mice were vaccinated with CTGF/E7 DNA vaccine, boosted one week later, and then subjected to lymphocyte subset depletion. Monoclonal Ab GK1.5 was used for CD4 depletion (Berkeley Antibody Company), monoclonal Ab 2.43 for CD8 depletion (Berkeley Antibody Company), and monoclonal Ab PK136 for Natural Killer 1.1⁺ depletion (Berkeley Antibody Company).

The monoclonal antibodies were injected intraperitoneally. One week after depletion, all groups of mice were challenged with 5×10⁴ TC-1 cells/mouse. Flow cytometric analysis revealed that 99% of these lymphocyte subsets were depleted while the other subsets were maintained at normal levels (data not shown). All mice were terminated on day 40 after the tumor challenge.

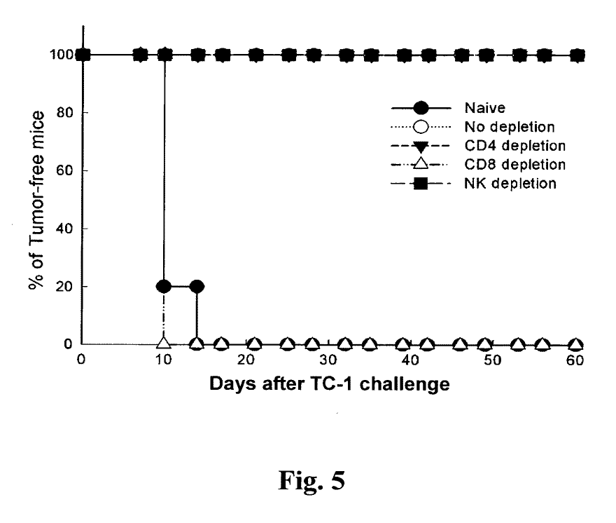

All naïve mice and all mice depleted of CD8⁺ T cells grew tumors within 14 days after TC-1 challenge. On the contrary, as shown in FIG. 5, all non-depleted mice and all vaccinated mice depleted of CD4⁺ T cells of natural killer 1.1⁺ cells remained tumor-free even 60 days after TC-1 tumor challenge. These results suggest that CD8⁺ T cells are important and essential for the anti-tumor immunity generated by the CTGF/E7 DNA vaccine.

In vivo Tumor Treatment Experiment

The therapeutic potential of the chimeric CTGF/E7 DNA vaccine was assessed by a lung hematogenous spread model because of its similarity to cancer metastasis (see Cheng et al., 2005). In this experiment, mice were injected with 5×10⁴ TC-1 cells via their tail veins. Two days later, each group of mice was immunized with pcDNA3 (no insert), pcDNA3-E7, pcDNA3-CTGF or pcDNA3-CTGF/E7 (16 μg/mouse), followed by a booster immunization every 7 days. The mice were sacrificed and their lungs were removed 28 days after TC-1 challenge. The pulmonary tumor nodules in each mouse were evaluated and counted by experimenters who were blind to the sample identity. The naïve group, in which the mice were not vaccinated, was used as a negative control.

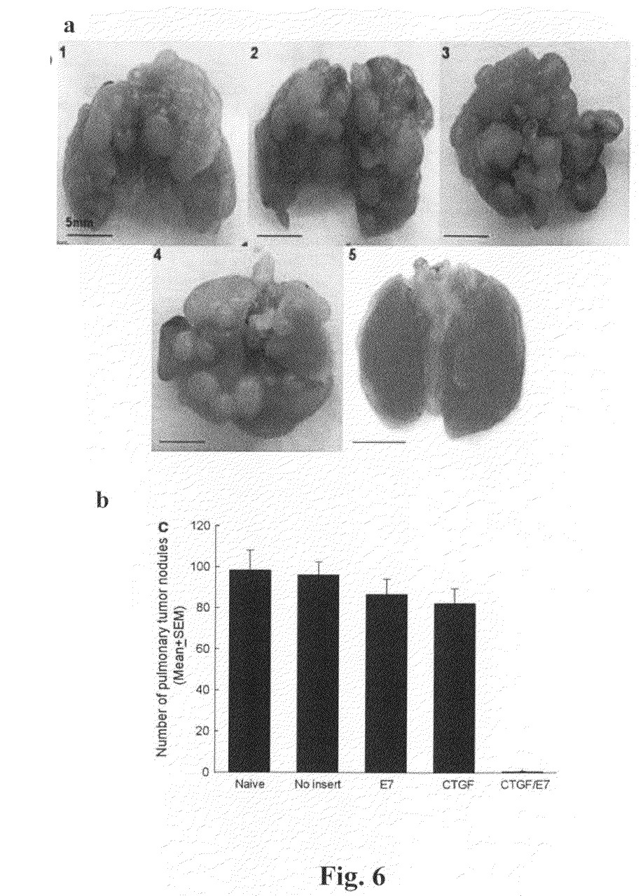

The representative pulmonary tumor nodules of each vaccinated mice group are shown in FIG. 6a, and the number of pulmonary tumor nodules of each vaccinated mice group are shown in FIG. 6b. Mice vaccinated with CTGF/E7 had significantly fewer pulmonary tumor nodules than all the other groups.

Preparation of CD11c⁺ Dendritic Cells in the Inguinal Lymph Nodes from Vaccinated Mice

Mice received an intradermal injection of pcDNA3-GFP, pcDNA3-E7/GFP or pcDNA3-CTGF/E7/GFP by a gene gun in the abdominal region. Inguinal lymph nodes of all mice were harvested 1 day or 5 days after vaccination. Dendritic cells, which express CD11c⁺ on the cell surface, were enriched from lymph nodes by CD11c (N418) microbeads (Miltenyi Biotec, Auburn, Calif.). Detection of apoptotic cells in the GFP⁺ CD11c⁺ cells was performed using the annexin V-PE apoptosis detection Kit-I (BD Bioscience, San Diego, Calif.) to count the percentage of apoptotic cells by flow cytometry. The naïve group, in which the mice were not vaccinated, was used as a negative control. In addition, 2×10⁴ above-mentioned enriched CD11c⁺ dendritic cells were incubated with 2×10⁶ E7-specific CD8⁺ T cell line (see Kim et al., 2003). These cells were then stained for both surface CD8 and intracellular IFN-γ, and then analyzed by flow cytometry as described.

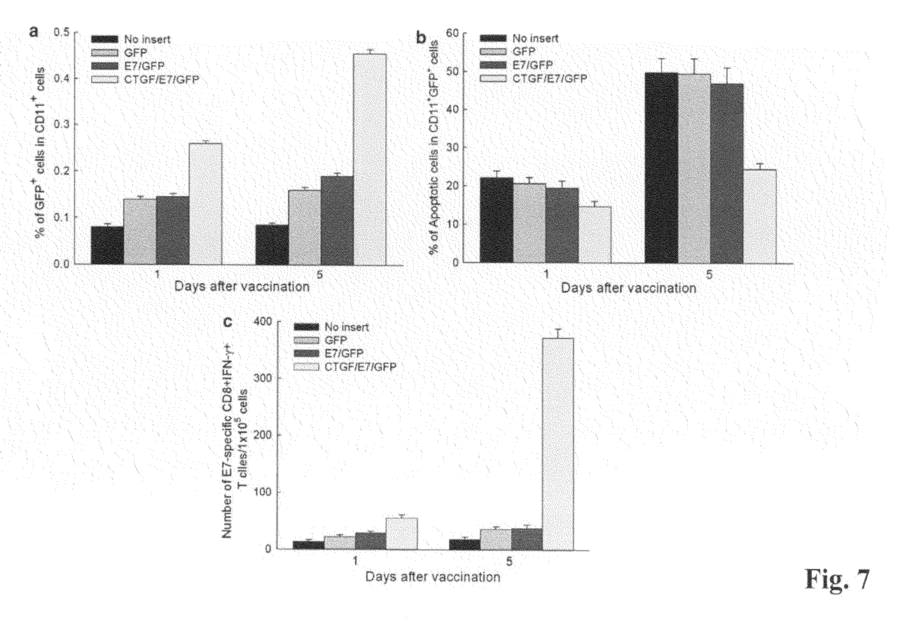

Results of the apoptosis detection kit are shown in FIGS. 7a-7c. As shown in FIG. 7a, there was no significant difference in the numbers of GFP⁺ CD11c⁺ cells in the inguinal lymph nodes at day 1 after vaccination. At day 5, however, we found a greater percentage of GFP⁺ CD11c⁺ cells in lymph nodes harvested from mice vaccinated with CTGF/E7/GEP than that of the groups E7/GFP and GFP only.

We further assayed the apoptotic cells in GFP⁺ CD11c⁺ cells derived form the inguinal lymph nodes of these groups. Mice vaccinated with DNA encoding CTGF/E7/GFP had a significantly lower percentage of apoptotic cells than mice vaccinated with GFP or E7/GFP, as shown in FIG. 7b. In other words, the apoptosis of dendritic cells is inhibited by CTGF/E7 vaccination.

The ability of the enriched CD11c⁺ dendritic cells to stimulate IFN-γ secretion was also evaluated. As shown in FIG. 7c, we compared the enriched CD11c⁺ dendritic cells 1 day and 5 days after gene gun vaccination. The CD11c⁺ dendritic cells isolated from the mice vaccinated with CTGF/E7/GFP were more effective in activating the E7-specific CD8⁺ T cell line to secrete IFN-γ than those isolated from the mice vaccinated with GFP and E7/GFP.

Since the E6 and E7 proteins represent the ideal targets for the development of HPV therapeutic vaccines, DNA vaccines targeting both E6 and E7 has the ability to generate a more potent immune response and anti-tumor effect than those targeting either E6 or E7 alone. Our previous studies also demonstrated that the vaccination with both DNA vaccines encoded E6 and E7 generates significantly better therapeutic anti-tumor effects against HPV E6- and E7-expressing tumors than vaccination with either E6 DNA or E7 DNA alone. These data suggest that it may be desirable to target both E6 and E7 to generate a better therapeutic effect against E6/E7 expressing tumors.

### Statistical Analysis

All data expressed as mean±SEM are representative of at least two different experiments. Data for intracellular cytokine staining with flow cytometric analysis and tumor treatment experiments were evaluated by analysis of variance (ANOVA).

### REFERENCES

Boyle et al. (1998) Enhanced responses to a DNA vaccine encoding a fusion antigen that is directed to sites of immune induction. Nature 392: 408-411

Chen et al. (2000) Enhancement of DNA vaccine potency by linkage of antigen gene to an HSP70 gene. Cancer Res 60; 1035-1042

Cheng et al. (2001) Tumor-specific immunity and antiangiogenesis generated by a DNA vaccine encoding calreticulin linked to a tumor antigen. J Clin Invest 108: 669-678

Cheng et al. (2005) Characterization of DNA vaccines encoding the domains of calreticulin for their ability to elicit tumor-specific immunity and antiangiogenesis. Vaccine 23: 3864-3874

Kim et al. (2003) Enhancing DNA vaccine potency by coadministration of DNA encoding antiapoptotic proteins. J Clin Invest 112: 109-117

Kim et al. (2004) Enhancement of DNA vaccine potency by coadministration of a tumor antigen gene and DNA encoding serine protease inhibitor-6. Cancer Res 64: 400-405

Kim et al. (2005) Modification of professional antigen-presenting cells with small interfering RNA in vivo to enhance cancer vaccine potency. Cancer Res 65: 309-316

King et al. (1998) DNA vaccines with single-chain Fv fused to fragment C of tetanus toxin induce protective immunity against lymphoma and myeloma. Nat Med 4: 1281-1286

Klinman et al. (1997) Contribution of CpG motifs to the immunogenicity of DNA vaccines. J Immunol 158: 3635-3639

Rodriguez et al. (1997) DNA immunization: ubiquitination of a viral protein enhances cytotoxic T-lymphocyte induction and antiviral protection but abrogates antibody induction. J Virol 71; 8497-8503

Weiss et al. (1998) A plasmid encoding murine granulocyte-macrophage colony-stimulating factor increase protection conferred by a malaria DNA vaccine. J Immunol 161: 2325-2332

What is claimed is:

1. A DNA construct, comprising:
an expression vector, which is expressible in a eukaryotic cell; and
a nucleotide fragment, which comprises a CTGF-encoding sequence, and a HPV sequence selected from an E6-encoding sequence, an E7-encoding sequence, or a combination thereof.

2. The DNA construct according to claim 1, wherein said expression vector is expressible in human cells.

3. The DNA construct according to claim 2, wherein said expression vector is selected from pcDNA3, pSG5, or pCMV.

4. The DNA construct according to claim 3, wherein said expression vector is pcDNA3.

5. The DNA construct according to claim 1, wherein said CTGF-encoding sequence is SEQ ID NO: 3.

6. The DNA construct according to claim 1, wherein said E6-encoding sequence is SEQ ID NO: 6.

7. The DNA construct according to claim 1, wherein said E7-encoding sequence is SEQ ID NO: 9.

8. A DNA vaccine, comprising:
the DNA construct according to claim 1; and
a particle, which is coated with said DNA construct.

9. The DNA vaccine according to claim 8, wherein said particle is a gold particle.

10. The DNA vaccine according to claim 9, said gold particle has a diameter of 1.6 μm.

11. The DNA vaccine according to claim 8, wherein said expression vector is expressible in human cells.

12. The DNA vaccine according to claim 9, wherein said expression vector is selected from pcDNA3, pSG5, or pCMV.

13. The DNA vaccine according to claim 12, wherein said expression vector is pcDNA3.

14. The DNA vaccine according to claim 8, wherein said CTGF-encoding sequence is SEQ ID NO: 3.

15. The DNA vaccine according to claim 8, wherein said E6-encoding sequence is SEQ ID NO: 6.

16. The DNA vaccine according to claim 8, wherein said E7-encoding sequence is SEQ ID NO: 9.

17. A pharmaceutical composition, comprising the DNA vaccine according to claim 8.

18. The pharmaceutical composition according to claim 17, further comprising a pharmaceutically acceptable carrier.

19. The pharmaceutical composition according to claim 17, which is used to treat HPV-induced diseases.

20. The pharmaceutical composition according to claim 19, which is used to treat cervical cancer, vaginal cancer, vulvar cancer, or penile cancer.

21. The pharmaceutical composition according to claim 20, which is used to treat cervical cancer.

22. The pharmaceutical composition according to claim 19, which is used to treat a cervical, vulvar, or vaginal precancerous lesion.

23. A method of generating the DNA vaccine of claim 8, comprising:
(1) providing a DNA construct comprising
an expression vector, which is expressible in a eukaryotic cell; and
a nucleotide fragment, which comprises a CTGF-encoding sequence, and a HPV sequence selected from an E6-encoding sequence, an E7-encoding sequence, or a combination thereof; and
(2) coating said DNA construct on the surface of a particle.

24. The method according to claim 23, wherein said expression vector is pcDNA3.

25. The method according to claim 23, wherein said CTGF-encoding sequence is SEQ ID NO: 3.

26. The method according to claim 23, wherein said E6-encoding sequence is SEQ ID NO: 6.

27. The method according to claim 23, wherein said E7-encoding sequence is SEQ ID NO: 9.

28. The method according to claim 23, wherein said particle is a gold particle.

29. The method according to claim 28, said gold particle has a diameter of 1.6 μm.

30. A method for preventing or treating a HPV-induced disease, comprising: administering an effective amount of the DNA vaccine according to claim 8 or an effective amount of the pharmaceutical composition according to claim 17 to a subject having or at risk for developing said HPV-induced disease.

31. The method according to claim 30, wherein said HPV-induced disease is cervical cancer, vaginal cancer, vulvar cancer, or penile cancer.

32. The method according to claim 31, wherein said HPV-induced disease is cervical cancer.

33. The method according to claim 30, wherein said HPV-induced disease is a cervical, vulvar, or vaginal precancerous lesion.


SEQUENCE LISTING (12 sequences)

SEQ ID NO: 1 - DNA, 30 bp, Artificial
  primer
ccggtctagacaaccatgaccgccgccagt

SEQ ID NO: 2 - DNA, 29 bp, Artificial
  primer
ccggaattcgttcaagttccagtctaatg

SEQ ID NO: 3 - DNA, 1100 bp, Human
caaccatgaccgccgccagtatgggccccgtccgcgtcgccttcgtggtcctcctcgccctctgcagccggccggccgtcggccagaactgcagcgggccgtgccggtgcccggacgagccggcgccgcgctgcccggcgggcgtgagcctcgtgctggacggctgcggctgctgccgcgtctgcgccaagcagctgggcgagctgtgcaccgagcgcgacccctgcgacccgcacaagggcctcttctgtgacttcggctccccggccaaccgcaagatcggcgtgtgcaccgccaaagatggtgctccctgcatcttcggtggtacggtgtaccgcagcggagagtccttccagagcagctgcaagtaccagtgcacgtgcctggacggggcggtgggctgcatgcccctgtgcagcatggacgttcgtctgcccagccctgactgccccttcccgaggagggtcaagctgcccgggaaatgctgcgaggagtgggtgtgtgacgagcccaaggaccaaaccgtggttgggcctgccctcgcggcttaccgactggaagacacgtttggcccagacccaactatgattagagccaactgcctggtccagaccacagagtggagcgcctgttccaagacctgtgggatgggcatctccacccgggttaccaatgacaacgcctcctgcaggctagagaagcagagccgcctgtgcatggtcaggccttgcgaagctgacctggaagagaacattaagaagggcaaaaagtgcatccgtactcccaaaatctccaagcctatcaagtttgagctttctggctgcaccagcatgaagacataccgagctaaattctgtggagtatgtaccgacggccgatgctgcaccccccacagaaccaccaccctgccggtggagttcaagtgccctgacggcgaggtcatgaagaagaacatgatgttcatcaagacctgtgcctgccattacaactgtcccggagacaatgacatctttgaatcgctgtactacaggaagatgtacggagacatggcatgaagccagagagtgagagacattaactcattagactggaacttgaac

SEQ ID NO: 4 - DNA, 34 bp, Artificial
  primer
ggggaattcatgcaccaaaagagaactgcaatgt

SEQ ID NO: 5 - DNA, 36 bp, Artificial
  primer
cccaagcttttacagctgggtttctctacgtgttct

SEQ ID NO: 6 - DNA, 477 bp, human papillomavirus 16
atgcaccaaaagagaactgcaatgtttcaggacccacaggagcgacccagaaagttaccacatttatgcacagagctgcaaacaactatacatgatataatattagaatgtgtgtactgcaagcaacagttactgcgacgtgaggtatatgactttgcttttcgggatttatgcatagtatatagagatgggaatccatatgcagtgtgtgataaatgtttaaagttttattctaaaattagtgagtatagatattattgttatagtgtgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcgagaacacgtagagaaacccagctgtaa

SEQ ID NO: 7 - DNA, 30 bp, Artificial
  primer
ccggaagcttatgcatggagatacacctac

SEQ ID NO: 8 - DNA, 23 bp, Artificial
  primer
cccaagcttttgagaacagatgg

SEQ ID NO: 9 - DNA, 288 bp, human papillomavirus 16
atgcatggagatacacctacattgcatgaatatatgttagatttgcaaccagagacaactgatctctactgttatgagcaattaaatgacagctcagaggaggaagatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcaa

SEQ ID NO: 10 - protein, 9 aa, human papillomavirus 16
RAHYNIVTF

SEQ ID NO: 11 - protein, 38 aa, human papillomavirus 16
DSSEEEDEIDGPAGQAEPDRAHYNIVTFCCKCDSTLRL

SEQ ID NO: 12 - protein, 8 aa, human papillomavirus 16
LCIVYRDG